(19) United States
(12) Patent Application Publication
(10) Pub. No.: US 2017/0172958 A1
(43) Pub. Date: Jun. 22, 2017

(54) Compounds for Use in Controlling Body Fat

(71) Applicant: Helga Refsum, Oslo (NO)

(72) Inventors: Amany Elshorbagy, Oxfordshire (GB); Helga Refsum, Oxfordshire (GB)

(21) Appl. No.: 15/123,909

(22) PCT Filed: Mar. 6, 2015
(86) PCT No.: PCT/GB2015/050662

§ 371 Date: Sep. 6, 2016

(30) Foreign Application Priority Data
Mar. 6, 2014 (GB) 1403969.7

(51) Int. Cl. A61K 31/198; A61K 31/255

(52) U.S. Cl. CPC A61K 31/198; A61K 31/255

(57) ABSTRACT

The invention relates to compounds that have utility in decreasing body fat of for preventing or decreasing the accumulation of body fat in a subject, and which inhibit the enzyme stearoyl-coenzymeA desaturase (SCD). The compounds have Formula I; where; R¹ is a C₁ to C₈ linear, branched or cyclic alkyl or alkenyl group having up to two double bonds, and optionally substituted with one or more groups selected from (i) one or more halogen atoms; (ii) alkoxy group of formula OR⁶; (iii) hydroxy; and (iv) carboxyl group of formula COOR⁷; R², R², R³, R³ are independently and for each occurrence selected from (a) hydrogen; (b) C₁ to C₄ alkyl optionally substituted with one or more groups selected from (i) to (iii) above; (c) halogen; and (d) C₁-C₂ alkoxy. Where R² and R² are both selected from either (b) or (d), R² and R² can optionally be linked. Where R³ and R³ are both selected from either (b) or (d), R³ and R³ can optionally be linked; R⁴ and R⁵ are each independently selected from hydrogen and C₁ to C₄ alkyl optionally substituted with one or more groups selected from (i) to (iii) above; R⁶ is selected from linear, branched or cyclic C₁ to C₄ alkyl optionally substituted with one or more halogen atoms; R7 is selected from hydrogen and linear, branched or cyclic C₁ to C₄ alkyl; and x is an integer from 1 to 3.

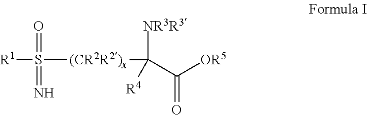

### FIELD OF THE INVENTION

The present invention is directed to compounds that have utility in decreasing body fat or for preventing or decreasing the accumulation of body fat. The compounds can be used to treat overweight or obese subjects, and to prevent or treat conditions associated with being obese or overweight.

### BACKGROUND TO THE INVENTION

Obesity is an increasingly important public health concern in both developed and developing countries, and is linked with a number of health conditions, including increased risk of diabetes, heart disease, osteoarthritis and some cancers. It can also negatively impact quality of life.

Obesity and increased cholesterol and fat levels can be controlled through diet management and exercise regimens, although these require long term commitment from a patient and are often unsuccessful.

Surgical treatments exist for obesity, for example liposuction, gastric banding and bariatric surgery. However, surgical interventions can carry the risk of infection, and unexpected complications. Additionally, patients require a subsequent lifestyle change to avoid recurrence.

Medications have been developed, for example orlistat (EP 0 129 748) and sibutramine (WO 98/13034). Orlistat inhibits pancreatic lipase, which prevents hydrolysis of triglycerides into absorbable free fatty acids, causing the triglycerides to go through the gut undigested. Negative side-effects include loose stools, faecal incontinence, frequent or urgent bowel movements, and flatulence. Sibutramine is a serotonin-norepinephrine reuptake inhibitor, and controls hunger by inducing a feeling of satiety. Associated side-effects include increased risk of adverse cardiovascular events, including heart attack and stroke.

Glutathione depletion has been implicated in reducing diet-induced weight gain (Kendig et al, Toxicology and Applied Pharmacology, 257, 2011, 338-348), where mice lacking the gene encoding for the modifier subunit (GCLM) of the enzyme glutamate-cysteine ligase (GCL) were shown to be resistant to weight gain compared to wild-type mice when fed a high-fat diet. GCL catalyses the formation of y-glutamyl cysteine from glutamate and cysteine which, in turn and in the presence of glycine produces glutathione.

The compound BSO (buthionine sulfoximine) is a glutathione biosynthesis inhibitor, and inhibits GCL. It has previously undergone phase I trials as a chemotherapeutic agent (e.g. Bailey et al, J. Clin. Onc., 1994, 12 (1), 194-205). Findeisen et al in Obesity, 19(2), 2011, 2429-2432 reported that BSO can increase energy expenditure and locomotor activity in mice, and reduce diet-induced obesity. The effects were attributed to glutathione depletion. However, Vigilanza et al in Journal of Cellular Physiology, 226, 2011, 2016-2024, reported that although BSO decreases intracellular glutathione, it resulted in triglyceride accumulation in adipocytes and increased adipogenesis. Therefore, it has not been clearly established that BSO reduces fat synthesis via its effect on glutathione.

### SUMMARY OF THE INVENTION

According to the present invention, there is provided a compound for use in reducing body fat or for preventing or reducing the accumulation of body fat in a subject, which compound is selected from those having Formula I, and pharmaceutically acceptable salts thereof, and wherein the compound inhibits the enzyme stearoyl-coenzymeA desaturase (SCD);

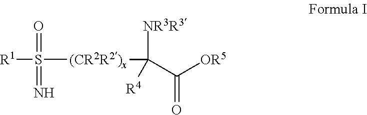

R¹ is a C₁ to C₈ linear, branched or cyclic alkyl or alkenyl group having up to two double bonds, and optionally substituted with one or more groups selected from (i) one or more halogen atoms; (ii) alkoxy group of formula OR⁶; (iii) hydroxy; and (iv) carboxyl group of formula COOR⁷.

R², R²′, R³, R³′ are independently and for each occurrence selected from (a) hydrogen; (b) C₁ to C₄ alkyl optionally substituted with one or more groups selected from (i) to (iii) above; (c) halogen; and (d) C₁-C₂ alkoxy. Where R² and R²′ are both selected from either (b) or (d), R² and R²′can optionally be linked. Where R³ and R³′ are both selected from either (b) or (d), R³ and R³′can optionally be linked.

R⁴ and R⁵ are each independently selected from hydrogen and C₁ to C₄ alkyl optionally substituted with one or more groups selected from (i) to (iii) above.

R⁶ is selected from linear, branched or cyclic alkyl optionally substituted with one or more halogen atoms;

R⁷ is selected from hydrogen and linear, branched or cyclic C₁ to C₄ alkyl.

x is an integer from 1 to 3.

There is also provided a method for reducing body fat or for preventing or reducing the accumulation of body fat in a subject, comprising administering to a subject in need thereof a therapeutically effective amount of a compound selected from those having formula I, and pharmaceutically acceptable salts thereof, and wherein the compound inhibits the enzyme SCD.

### DETAILED DESCRIPTION OF THE INVENTION

The compounds and salts of Formula I help to control body fat. In particular they can be used to reduce body fat, or to prevent or reduce the accumulation of body fat in a subject. They can be used to treat subjects who are obese or overweight. They can also be used to prevent or treat conditions associated with being obese or overweight.

The compounds and salts of Formula I can also be used to avoid or decrease accumulation of body fat. This can be useful for subjects who are susceptible to gaining weight, for example subjects who have undergone treatment for being obese or overweight, or subjects who suffer from cardiovascular disease (for example congestive heart failure, hypertension and atherosclerotic disease) and diabetes mellitus, and also subjects who are predisposed to certain forms of cancer, e.g. breast cancer and prostate cancer.

The enzyme SCD is involved in the synthesis, storage and accumulation of lipids in liver and in adipocytes. Therefore, lowering the activity of SCD can limit the accumulation of lipids in the liver and in the adipose tissue. Avoiding or reducing the formation of adipose tissue by preventing differentiation of preadipocytes into mature adipocytes can be beneficial to subjects at critical stages of body development, particularly in children. This can help to reduce the chances of onset of obesity later in life.

An advantage of the present invention is that the compounds or salts of Formula I are highly selective in reducing or avoiding accumulation of fat, with little effect on other body tissues such as muscle or bone.

Conditions associated with being obese or overweight include coronary heart disease, angina, high blood pressure, type 2 diabetes, glucose intolerance, insulin resistance, stroke, cancer (including cancer of the oesophagus, pancreas, colon, rectum, breast, endometrium, kidney, thyroid, gallbladder), infertility, depression, liver disease (such as non-alcoholic fatty liver disease and liver cirrhosis), kidney disease (such as chronic renal failure), dementia, osteoarthritis, gastro-oesophageal reflux disease and sleep apnoea.

When administered to a subject, the compounds and salts of Formula I ensure a high level of insulin sensitivity even when the subject is being fed a high fat diet. This means that they can be useful in treating insulin resistance and in treating type 2 diabetes. This is contrary to the observations of Ogihara et a/. in Diabetologica (2005), 47, pp794-805, which reported that BSO induces insulin resistance in rats. Although Findeisen et a/. (see above) reported that BSO could help to preserve insulin sensitivity, this was based on its purported effects on depleting endogenous glutathione, and not to SCD inhibition.

In the present invention, administration of the compound of Formula I results in the inhibition of SCD. SCD is membrane-bound in the endoplasmic reticulum, and catalyses desaturation of fatty acids through desaturation of the associated fatty-acyl coenzyme A. Examples are the conversion of stearic acid to oleic acid, and palmitic acid to palmitoleic acid. Two forms are known in humans, SCD-1 and SCD-5. In mice, four forms are known, SCD-1 to SCD-4. SCD deletion in rodents produces a lean, hypermetabolic phenotype. This is believed to result from diversion of unconverted fatty acids away from being converted into triglycerides, which would result in them being stored in adipose tissue, and instead to being broken down by beta-oxidation via activation of AMP kinase in the liver.

Increased SCD-1 activity indices in elderly humans has been associated with obesity and obesity-related diseases (Vinknes et al, Obesity, 21(3), 2013, E294-E302, and Warensjo et al, Diabetologica, 48, 2005, 1999-2005).

The inventors have now found that compounds or salts of Formula I, in particular BSO, can act to inhibit SCD. This is achieved inter alia by suppressing plasma tCys levels, which provides an alternative mechanism of controlling obesity and excess weight, and conditions associated with being obese or overweight.

Previously, any link between BSO and treating obesity was associated with its activity towards lowering cellular glutathione levels. Because synthesis of glutathione requires cysteine, lowering glutathione would not be expected to decrease the cysteine levels. Further, the present invention results in avoidance of triglyceride formation, contrary to the teaching of Vigilanza et al as discussed above.

Compounds of Formula I inhibit SCD activity, which can be measured using one or more activity indices. These can be based on measurements of different fatty acid components of the blood plasma or serum.

Activity indices are based on the amount of monounsaturated fatty acids relative to their respective unsaturated precursors. The most commonly used SCD activity indices are derived from the ratio of palmitoleic to palmitic acids (“SCD16 index”), and the ratio of oleic to stearic acid (“SCD18 index”). These ratios are lowered with increased inhibition of SCD. SCD indices can either be calculated from plasma/serum free fatty acids, or from the total fatty acid pool, which also includes esterified fatty acids (e.g. triglycerides and phospholipids). Indices calculated from free fatty acid concentrations reflect SCD activity in adipose tissue, while those from total fatty acids reflect SCD activity in liver (Warensjo et al, Lipids Health Dis 2009, 8:37). Both in rodents and humans, SCD indices are associated with fat mass.

Further, SCD activity increases with high tCys concentration in both rodents and humans. tCys refers to all natural forms of circulating cysteine, such as cysteine (thiol form), cystine (disulphide form), cysteine-mixed disulphides with other thiol compounds, and protein-bound cysteine not in peptide linkage. tCys in the plasma or serum can be measured using techniques described, for example, in WO2010/010383.

To assess the extent of the beneficial effects of the compounds of Formula I, values of the plasma/serum concentrations of tCys and/or of the SCD activity index in a subject before and after administration of a compound of Formula I can be compared. Additionally or alternatively, comparison can be made between a subject who has received a compound of Formula I, and one or more control subjects who have not received a compound of Formula I.

Other indices include the volume of oxygen consumed and/or the volume of carbon dioxide produced by a subject, typically normalised to body weight. Subjects who have been treated with a compound or salt according to Formula I tend to have increased utilisation of oxygen, and increased production of carbon dioxide, compared to the subject before administration of the compound according to Formula I and/or control subjects.

Performance targets include:
a) reduction of body weight (kg) and/or or body mass index, BMI (calculated as body weight (kg) /height (m) squared).
b) decrease in fat mass (kg) measured by dual energy X-ray absorptiometry, bioelectric impedance, CT or MRI scans.
c) reduction of body fat % (calculated as 100* (fat mass/total body weight)).
d) decrease in waist circumference (large waist circumference is associated with greater cardiometabolic risk).
e) decreased estimated hepatic desaturase activity (calculated from fatty acid profile).
f) improved adipokine profile and reduction in low grade inflammation.
g) reduction in non-alcoholic fatty liver disease.
h) reduction in obesity-related disorders (see above).

Activity can also be determined from plasma or liver glutathione (GSH) concentrations. Use of compounds or salts according to Formula I can result in lower concentrations of total glutathione (tGSH) in the plasma and the liver, lower reduced glutathione (rGSH) concentrations in plasma, and/or increased rGSH/tGSH ratios in the liver. Thus, compounds or salts of Formula I appear to lower the oxidised glutathione rather than the reduced form in the liver. Since reduced glutathione is an antioxidant, this can explain why the use of compounds or salts of Formula I can have beneficial effects on liver diseases, such as non-fatty liver disease and liver cirrhosis. It also means that a patient can benefit from the fat-controlling effects of compounds or salts of Formula I using doses lower than those used, for example, when treating diseases such as cancer.

The invention is preferably directed towards reducing body fat in subjects who are overweight or obese. The subject is preferably a mammal, more preferably a human. In a human, being overweight is typically associated with individuals having a BMI of 25 kg/m² or more. Being obese is typically associated with those who have a BMI of 30 kg/m² or more. The invention is also useful for human subjects who have a BMI of 28 kg/m² or more, and have associated risk factors, for example the conditions associated with being obese described above.

Alternatively, the invention can be directed towards avoiding or reducing accumulation of body fat in a subject, the subject preferably being a mammal, and more preferably a human.

The compound or salt of Formula I can be administered by various means, in the form of a pharmaceutical composition, which preferably takes the form of therapeutically effective individual doses of the compound of Formula I or salt thereof, adjusted to the form of administration. Administration can be by various means, for example oral, enteral or parenteral.

For oral administration, the composition can be formulated into solid or liquid preparations, such as pills, tablets, troches, capsules, powder, granules, syrups, solutions, suspensions or emulsions. In another embodiment, it can be mixed with food or drink, for example being part of a nutritional formulation or composition provided for the subject as part of a managed dietary regime.

Solid compositions can comprise one or more of the following in addition to the desired quantity of the compound of Formula I or salt thereof: a pharmaceutically active carrier, including conventional ingredients such as lactose, sucrose and cornstarch; binders such as acacia, cornstarch or gelatine; disintegrating agents, such as potato starch or alginic acid; and lubricants such as stearic acid or magnesium stearate. Optionally, the pharmaceutical composition can be a sustained release formulation, in which the compound according to Formula I, or salt thereof, is incorporated in a matrix of an acrylic polymer or chitin, for example.

Examples of liquid compositions for oral administration include aqueous solutions such as syrups, flavoured syrups, aqueous or oil suspensions, optionally flavoured emulsions with edible oils, and elixirs. Suspensions can include dispersing or suspending agents such as synthetic and natural gums, for example tragacanth, acacia, alginate, dextran, sodium carboxymethylcellulose, methylcellulose, polyvinylpyrrolidione and gelatin.

For parenterally-administered compositions, the compound of Formula I or salt thereof is typically formulated with a suitable liquid injection vehicle, which include for example water, saline, dextrose, water-miscible solvents such as ethanol, polyethylene glycol and propylene glycol, and non-aqueous vehicles such as plant or animal oils. Optionally, the medicament can be an emulsion. Optionally, the pH is in the range from 6 to 8, preferably 6.5 to 7.5. Optionally, buffers such as citrates, acetates or phosphates, can be present. Optionally, antioxidants such as ascorbic acid or sodium bisulphite can be present.

Optionally, solubilising agents and stabilisers such as cyclodextrin, lysolecithin, oleic acid, stearic acid, and dextrin can be present. Optionally, local anaesthetics such as lignocaine and procaine hydrochloride can be present. Parenteral administration can be, for example, intramuscular, intravenous, intradermal or subcutaneous.

Suitable doses of the compound of Formula I or salt thereof are in the range of from 0.1 to 100 mmol per kg body mass per day, or 0.025 to 25 g per kg body mass per day. In some embodiments, for humans, the dose is in the range of from 25 to 450 mg per kg body mass per day, for example 25 to 250 mg per kg body mass per day. In further embodiments, the dose for humans is in a range of 0.9 to 17 g/m²/day, for example 0.9 to 9 g/m²/day.

The compound of Formula I or salt thereof can be provided in one dose, or more than one dose, typically in the range of from one to eight doses per day. Preferably, a minimum of two doses per day are taken, for example from two to four or from two to three doses per day.

The compounds or salts according to Formula I can be administered in combination with one or more additional compounds that are effective for use in treating medical conditions. For example, the compounds or salts of Formula I can be administered in combination with one or more additional compounds that are effective for use in treating subjects who are obese or overweight, or conditions associated with being obese or overweight as described above. In one embodiment, two or more compounds of Formula I can be administered in combination. Combination treatment can involve administering the different compounds separately, simultaneously or sequentially. The two or more compounds can be provided in the form of a kit comprising separate pharmaceutical compositions for each compound. Alternatively, two or more compounds can be incorporated into a single pharmaceutical composition.

The compounds of Formula I can be synthesised by known means, or purchased from commercial suppliers. Examples of synthetic procedures can be found in U.S. Pat. No. 5,476,966, by Hiratake et al in Biosci. Biotechnol. Biochem., 66(7), 2002, 1500-1514, and by Tokutake et al in Bioorg. Med. Chem., 6, 1998, 1935-1953.

In the compounds of Formula I, any halide substituent is preferably F or Cl.

Optional substituents on any alkyl, alkenyl or alkoxy group are preferably halide, preferably F or Cl.

In preferred embodiments, R¹ has no more than 2 optional substituents. Preferably, R¹ is a C₃ to C₅ linear, branched or cyclic alkyl, which is more preferably non-substituted. R¹ is more preferably a non-substituted C₄ alkyl, preferably n-butyl.

x is preferably 2.

In preferred embodiments, there are no more than two optional substituents on each of R² and R²′. Preferably, R² and R²′ are independently and for each occurrence selected from hydrogen and optionally substituted C₁₋₂ alkyl. In a further embodiment, all R² groups are hydrogen, and no more than two R²′ groups are other than hydrogen, which are preferably selected from optionally substituted C₁₋₂ alkyl. Preferably no more than one R²′ group is other than hydrogen. In a preferred embodiment, all R² and R²′ groups are hydrogen. Preferably, (CR²R²′)ₓ is (CH₂)₂.

R³ and R³′ each preferably contains no more than two optional substituents. R³ and R³′ are each independently preferably selected from hydrogen and optionally substituted C₁₋₂ alkyl. Preferably, R³ is hydrogen and R³ is hydrogen or non-substituted C₁₋₂ alkyl. In a preferred embodiment, both R³ and R³′ are hydrogen.

Preferably, there are no more than two optional substituents on each of R⁴ and R⁵. Each of R⁴ or R⁵ is preferably hydrogen or optionally substituted C₁₋₂ alkyl. More preferably, each or both of R⁴ and R⁵ are selected from hydrogen and non-substituted C₁₋₂ alkyl. Most preferably, R⁴ and R⁵ are both hydrogen.

Preferably, the compound is buthionine sulfoximine (BSO) or a pharmaceutically acceptable salt thereof.

The compounds or salts of Formula I can be used as a racemic mixture or in an enantiomerically purified form. The L-isomer is preferred (e.g. L-buthionine-sulfoximine).

A pharmaceutically acceptable salt of any of the compounds of Formula I includes, for example, sodium or potassium salts. Further examples include salts based on a quaternary—NR³R³′ group, e.g. —[NR³R³′R³″]X. r³″ can be as defined above for R³ and R³′, and can optionally be linked with either or both of R³ and R³′ where R³″ and at least one of R³ and R³′ are selected from (b) and (d). Examples of X include halide, hydroxide, nitrate, sulphate and carbonate.

The compounds and salts of Formula I are able to inhibit the formation of adipose tissue, inhibit body fat formation, inhibit fatty acid concentrations in the plasma, and also inhibit the unsaturation of fatty acids. This helps to advance weight loss through reduction in fatty deposits and adipose tissue. This is achieved by inhibiting the enzyme SCD, which inhibits fatty acid biosynthesis, and which decreases the production and storage of fatty deposits and adipose tissue.

The compounds and salts of Formula I are also specific to the reduction in white adipose tissue, compared for example to brown adipose tissue (BAT) and lean, muscular tissue. Since obesity and being overweight is typically associated with excess white adipose tissue, the compounds and salts of Formula I can be highly specific in helping to reduce weight in overweight and obese people without negative effects associated with loss of muscular tissue and BAT. BAT is often referred to as “beneficial” fat. It protects against obesity via uncoupling protein 1 (UCP1), which helps convert energy from food into heat, rather than store it as fat. Thus the lack of significant reduction of BAT by the compounds and salts of Formula I suggests a favorable specific effect on white fat, without compromising brown fat.

### DRAWINGS

Unless otherwise indicated, line graphs and bar charts show mean±SEM.

FIG. 1 is a graph showing the change in mean body weight with time for control mice and BSO-treated mice on a high fat diet.

FIG. 2 is a bar chart showing the total lean and total fat mass in control mice and BSO-treated mice, as determined using echo MRI.

FIG. 3 is a bar chart showing the percentage of lean versus fat mass in control and BSO-treated mice, based on echo MRI.

FIG. 4 is a bar chart showing the amount of abdominal fat (g) in control and BSO-treated mice.

FIG. 5 is a bar chart comparing the amount and distribution of brown fat mass (g) in control and BSO-treated mice.

FIG. 6 is a collection of bar charts which compare oxygen consumption and carbon dioxide production of control and BSO-treated mice, normalised to body weight.

FIG. 7 is a bar chart comparing the concentration of amino acids in the plasma of control and BSO-treated mice.

FIG. 8 is a bar chart comparing the plasma tCys concentrations in control and BSO-treated mice.

FIG. 9 is a bar chart comparing the plasma homocysteine concentrations in control and BSO-treated mice.

FIG. 10 is a bar chart comparing the plasma glutathione concentrations in control and BSO-treated mice.

FIG. 11 is a bar chart comparing the effects of BSO treatment on the plasma concentrations of various compounds involved in the formation or utilisation of cysteine.

FIG. 12 schematically illustrates the pathways involved in cysteine and glutathione synthesis, and the respective precursors and products formed.

FIG. 13 is a bar chart comparing concentrations of various protein-related components of blood plasma in control and BSO-treated mice.

FIG. 14 is a bar chart comparing fatty acid-related components of blood plasma in control and BSO-treated mice.

FIG. 15 is a bar chart comparing plasma concentrations of glucose and glycerol in control and BSO-treated mice.

FIG. 16 is a bar chart comparing the free palmitic acid and palmitoleic acid concentrations in the plasma of control and BSO-treated mice.

FIG. 17 is a bar chart comparing the free stearic acid and oleic acid concentrations in the plasma of control and BSO-treated mice.

FIG. 18 is a bar chart comparing the free fatty acid plasma concentrations of myristic acid, linolenic acid, y-linolenic acid, dihomo-y-linoleic acid, linoleic acid and arachidonic acid in control and BSO-treated mice.

FIG. 19 is a bar chart comparing the total fatty acid plasma concentrations of palmitic acid, stearic acid, oleic acid, linoleic acid arachidonic acid and docosahexaenoic acid in control and BSO-treated mice (means and SD).

FIG. 20 is a bar chart comparing the total fatty acid plasma concentrations of myristic acid, palmitoleic acid, y-linoleic acid, linolenic acid, dihomo-y-linoleic acid and eicosapentaenoic acid (means and SD).

FIG. 21 is a bar chart comparing the unsaturated to saturated total palmitoleic/palmitic acid and total oleic/stearic acid plasma concentration ratios in control and BSO-treated mice, otherwise called the SCD16 and SCD18 activity indices, respectively (means and SD).

FIG. 22 shows a comparison of fat vacuolation scores in liver cells between control and BSO-treated mice, with an example of the histology shown on the right.

FIG. 23 is a series of plots showing glucose plasma levels, insulin plasma levels and leptin plasma levels for BSO-fed mice and control mice. It also shows the HOMA-IR (homeostatic model of insulin resistance) index.

FIG. 24 shows the concentrations of reduced glutathione and total glutathione in the plasma and in the liver of BSO-fed and control mice. Reduced/total glutathione ratios

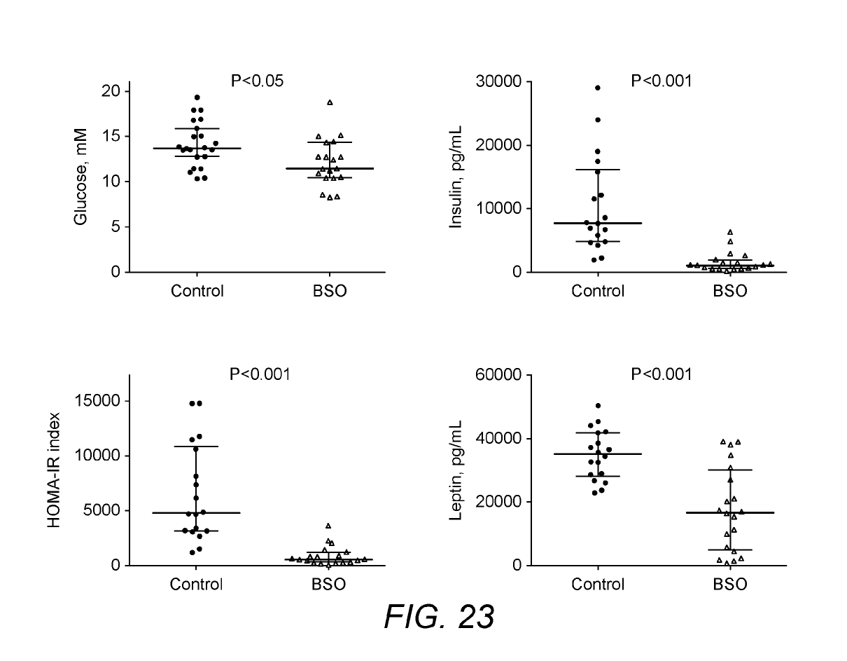

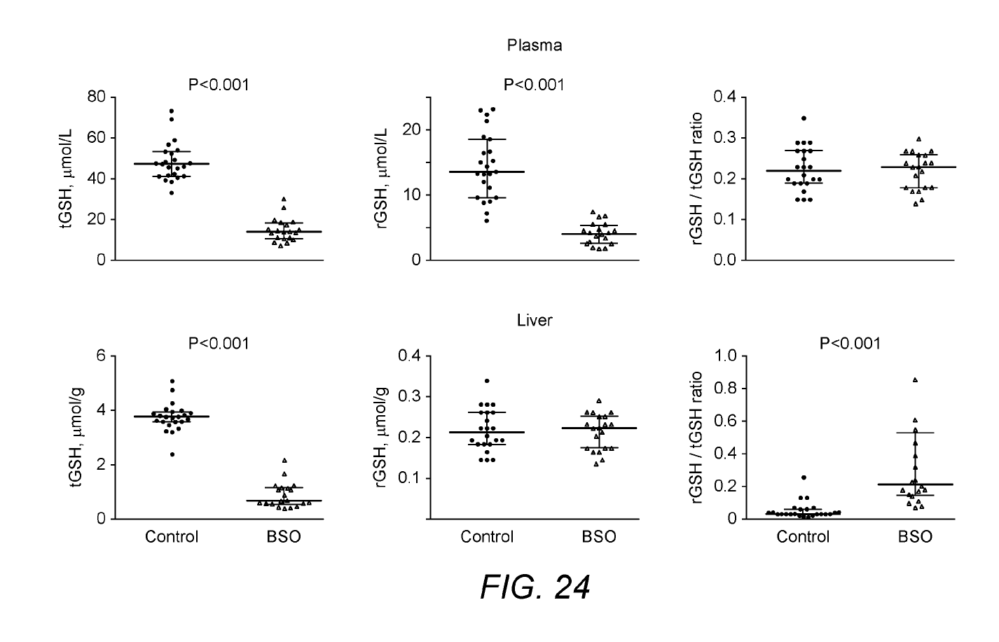

are also plotted. In FIGS. 23 and 24, the lines represent the median, 25^{th} and 75^{th} percentiles of the plotted data.

### EXAMPLES

Male C3H mice were used. Two cohorts of twelve mice were weaned and fed on a commercial standard diet (SDS Rat and Mouse No.3 Breeding diet (RM3)), containing 3.36 g% fat, 22.45 g % protein and 71.21 g % carbohydrate, till maturity. At 11 weeks of age, the standard diet was replaced with a high fat diet (Research Diets Inc, USA, D12492, containing 35 g % fat, from lard and soybean oil). One cohort was provided with BSO (30 mmol/L) in their drinking water, concomitant with the start of high fat feeding, while the other cohort (control group) received no treatment. The mice were kept in accordance with UK Home Office welfare guidelines and project license restrictions under controlled light (12-h light and 12-h dark cycle; dark 7 p.m.-7 a.m.), temperature (21° C.±2° C.) and humidity (55%±10%) conditions. They had free access to water (10 ppm chlorine) and food throughout the experiment.

Phenotyping tests were performed according to the European Phenotyping Resource for Standardised Screens (EMPReSS) from EUMORPHIA standardized protocols, available at http://empress.har.mrc.ac.uk. Body mass was measured daily on scales calibrated to 0.01 g. Metabolic rate was measured using indirect calorimetry (Oxymax; Columbus Instruments) to determine oxygen consumption and carbon dioxide production. Body composition (% fat tissue and % lean tissue) was assessed utilizing an EchoMRl-100 quantitative magnetic resonance whole body composition analyzer (Echo Medical Systems, Houston, Tex.).

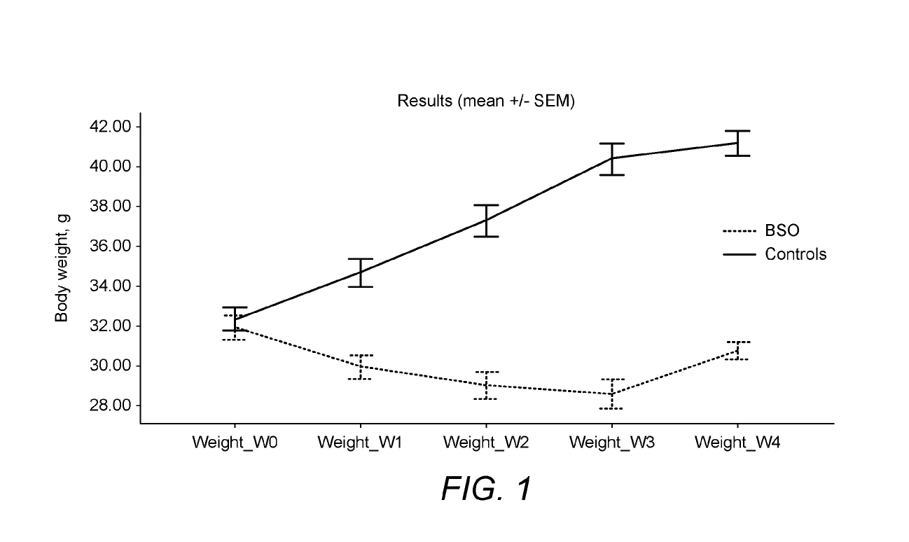

The BSO-treated group exhibited a reduction in body weight over the course of the four week treatment, as shown in FIG. 1, whereas the control group showed a significant weight gain.

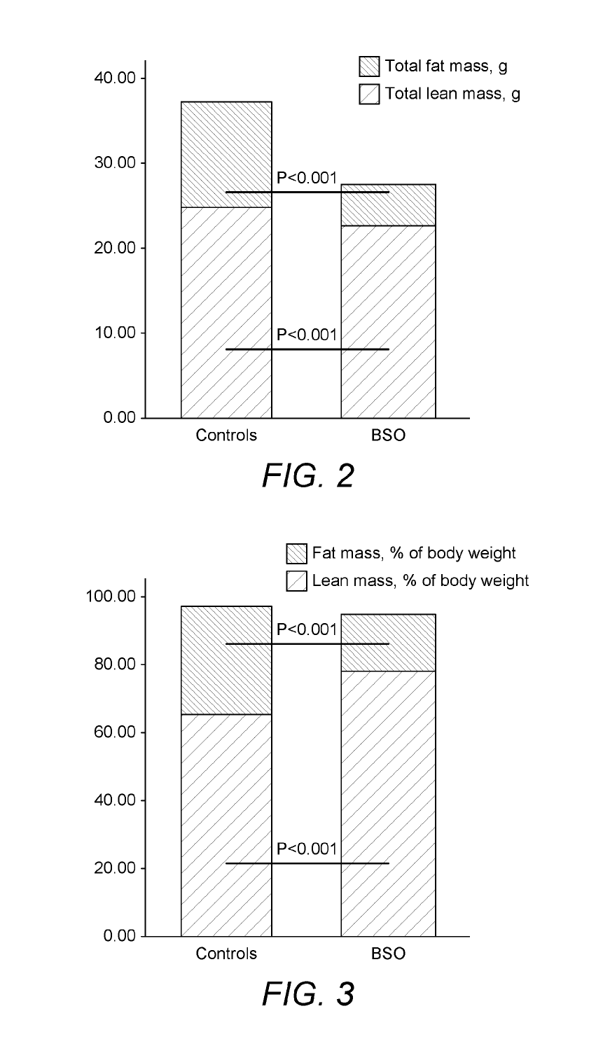

The quantity of body fat in the BSO-treated mice was substantially lower compared to the control mice, with only a relatively small difference in the lean mass between the two cohorts. The results are shown in FIGS. 2 and 3, demonstrating that the effects of BSO are highly selective to fat mass, as opposed to lean mass.

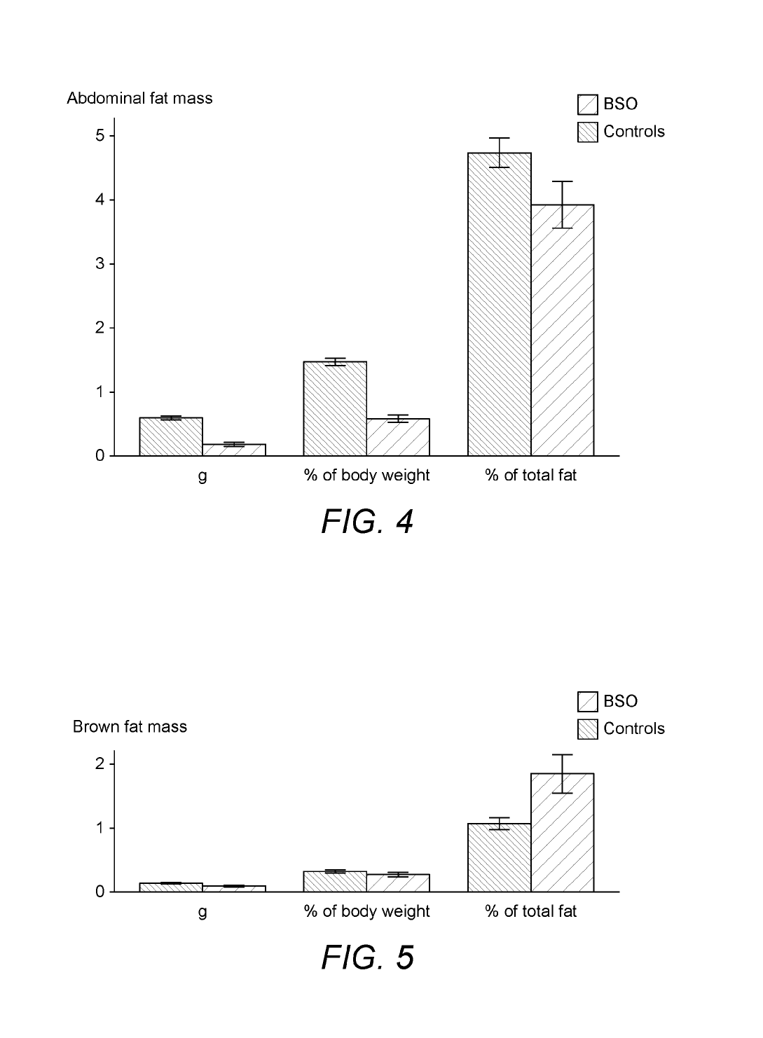

An analysis of abdominal fat in dissected mice shows that the quantity of abdominal fat in the BSO mice, in grams, and as a percentage of total body weight, is significantly lower than the control cohort (P<0.001 for both). The abdominal fat mass as a percentage of total fat mass also shows a trend towards decrease in BSO mice compared to control (P=0.055), as shown in FIG. 4. This demonstrates that there is an enhanced effect of BSO on abdominal fat mass compared to other fat depots.

FIG. 5 demonstrates that the amount of brown fat mass in BSO-treated mice (measured on dissected mice) is similar to the brown fat mass in the control mice, although as a percentage of total fat mass the percentage of brown fat in the BSO-treated mice is higher (P<0.05). This demonstrates that BSO has a particularly enhanced depleting effect on white fat (white adipose tissue), while sparing brown fat (BAT), which exerts an anti-obesity effect as explained earlier.

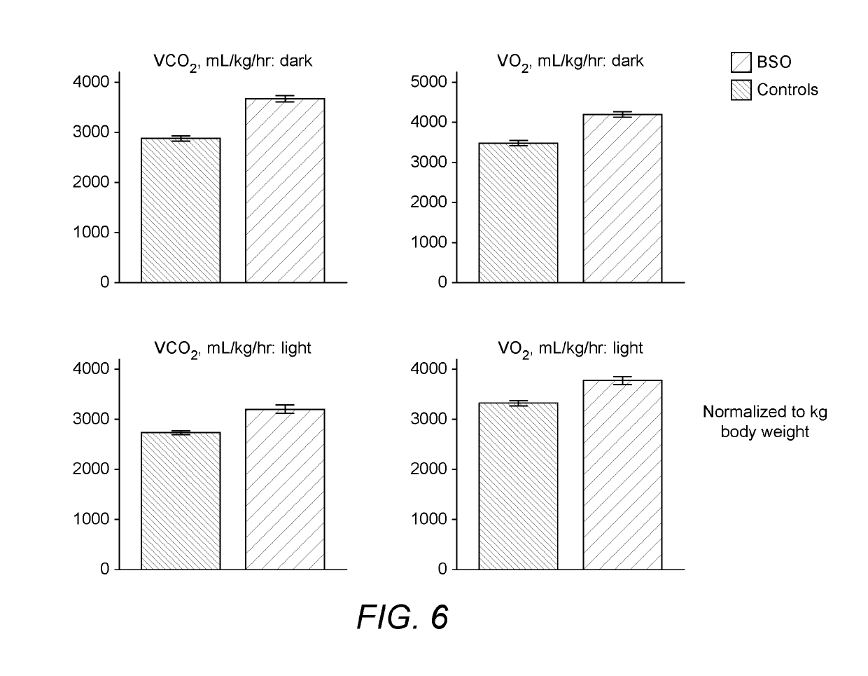

Oxygen consumption and carbon dioxide production were also measured, with results shown in FIG. 6. The BSO-treated mice showed significantly greater oxygen consumption in the dark and light phases (P<0.001 for both) and greater carbon dioxide production in the dark and light phases (P<0.001 for both) per unit weight. This is consistent with increased energy consumption, and hence decreased fat storage in the BSO-treated mice.

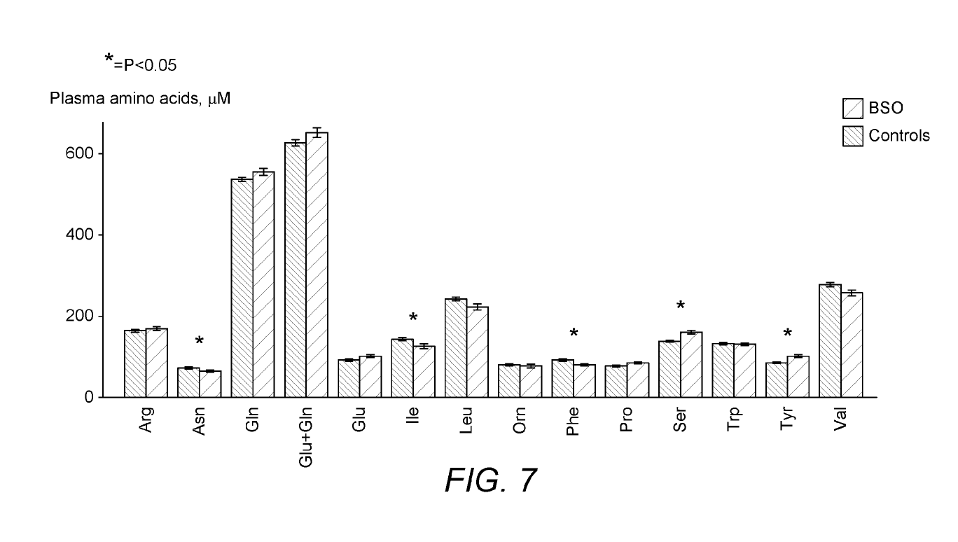

FIG. 7 shows the concentration of amino acids (in μ mol/L) in the plasma, in which there is little difference between the two cohorts, indicating that the effects of BSO on protein/lean tissue are minimal, and that the BSO effects result from a specific action on sulphur amino acid metabolism.

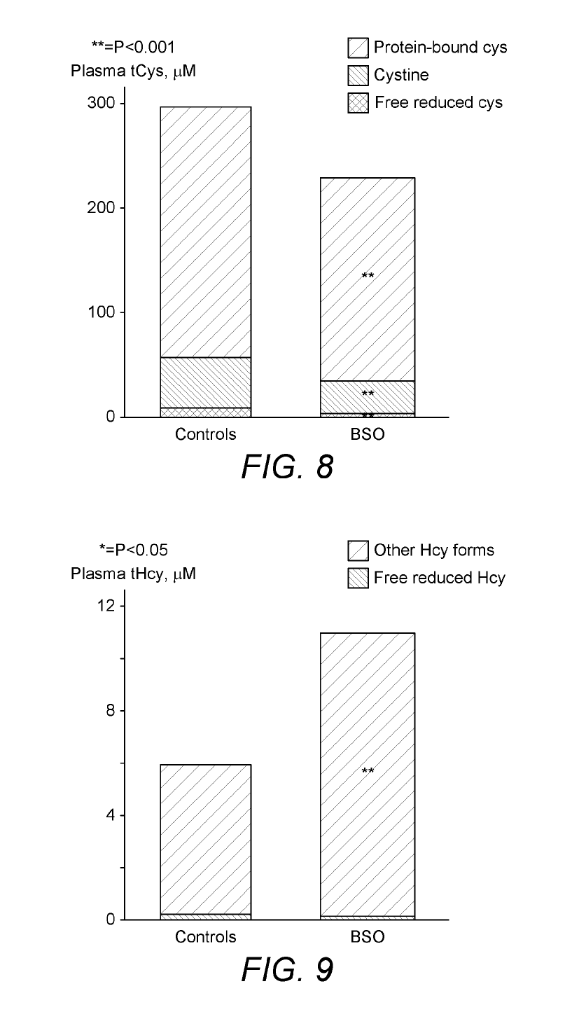

The effects of BSO on plasma tCys concentrations are shown in FIG. 8. The concentrations are significantly and substantially lower in the BSO-treated mice compared to controls. The effects are apparent for all the measured forms of cysteine.

Total homocysteine concentrations, tHcy (i.e. free-reduced homocysteine, homogeneous and mixed disulphides, and protein-bound homocysteine) were also measured, and are shown in FIG. 9. BSO-treated mice showed larger concentrations in the plasma compared to control, which would be consistent with a reduction in tCys concentrations, in that less tHcy or cystathionine is converted to cysteine.

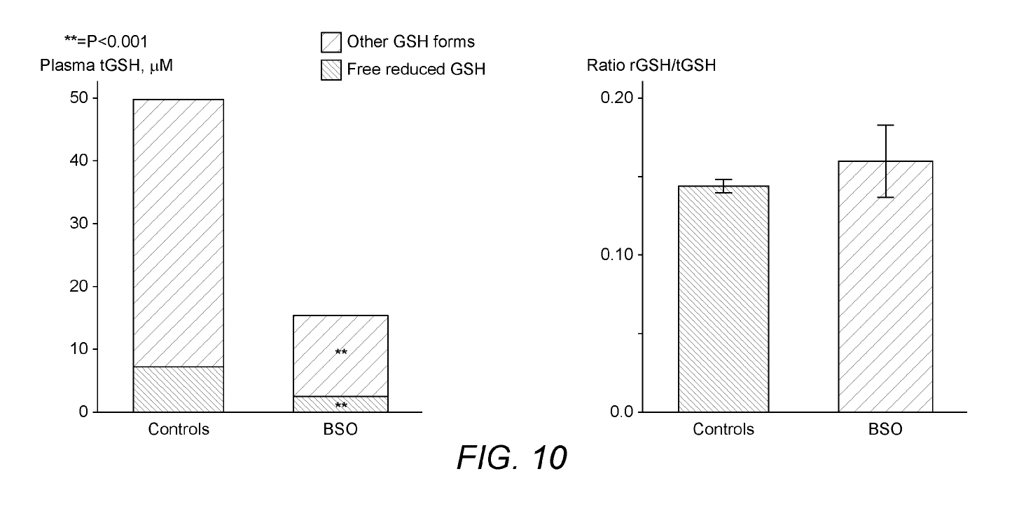

FIG. 10 confirms that one effect of BSO is to decrease plasma glutathione concentrations.

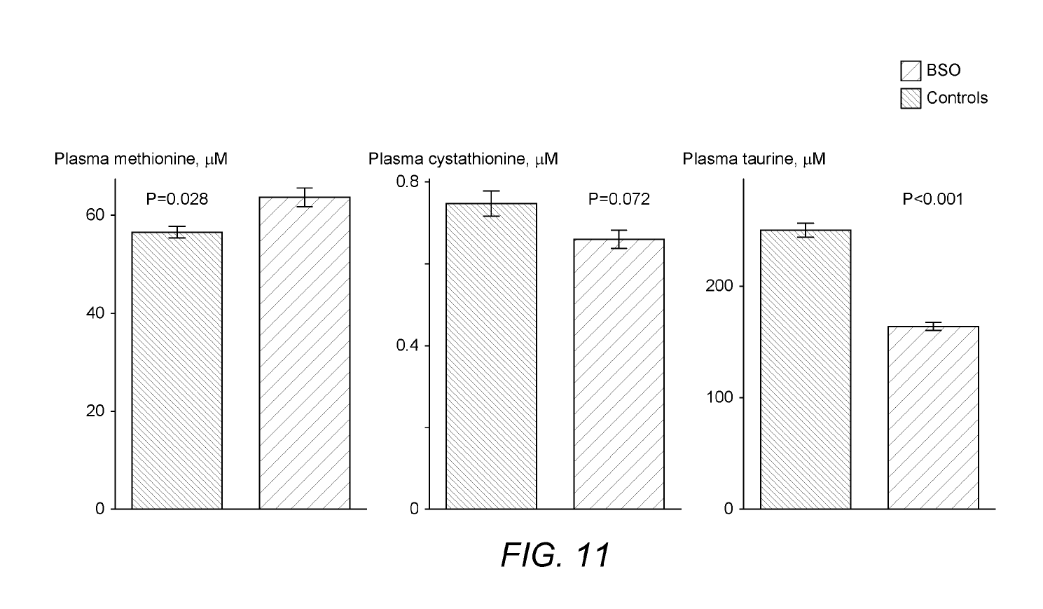

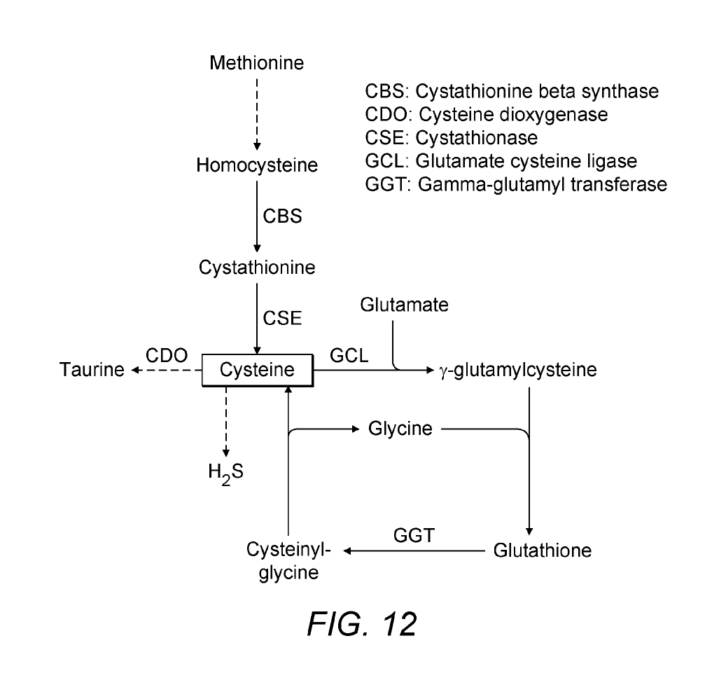

FIG. 11 shows the effects of BSO treatment on various compounds involved in the formation of cysteine. Because cysteine is converted to taurine by the enzyme cysteine dioxygenase, the substantially lower taurine concentrations compared to the control cohort are consistent with the low tCys levels in the plasma. FIG. 12 schematically illustrates the pathways involved in cysteine and glutathione synthesis, and the respective precursors and products formed. The dotted lines indicate pathways with omitted intermediates for clarity.

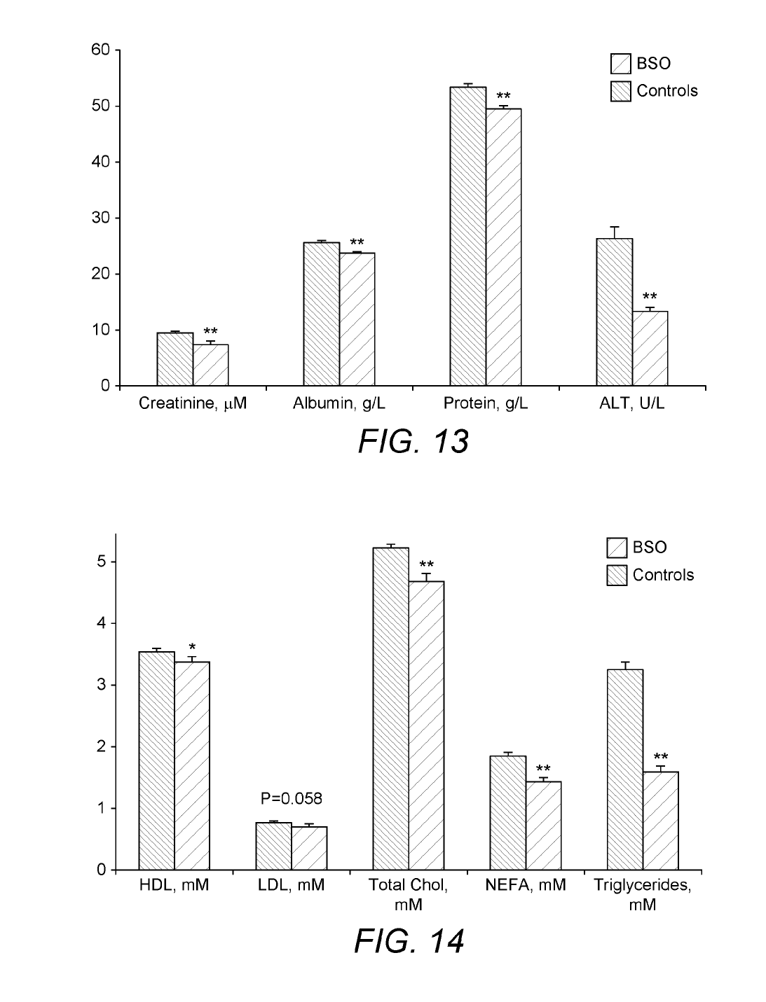

Plasma concentrations of creatinine, albumin, protein and ALT (a liver enzyme and marker of non-alcoholic fatty liver disease (NAFLD)) are shown in FIG. 13. The major difference between the two cohorts is in the ALT concentrations, with the BSO-treated mice having substantially lower concentrations compared to control. This indicates protection against liver insult due to NAFLD (as confirmed by liver histology results below), and lower risk for diabetes and metabolic syndrome (Schindhelm et al, Diab Metab Res Rev 2006; 22 (6), 437-443).

FIG. 14 compares differences in fat and lipid components of the plasma, namely high density lipoprotein (HDL), low density lipoprotein (LDL), total cholesterol, non-esterified fatty acids, and triglycerides. The concentrations are lower in the BSO-treated mice in all cases, although particularly so for the triglyceride concentrations. Elevated plasma triglycerides is an independent risk factor for myocardial infarction, ischemic heart disease and death in men and women (Nordestgaard et al, JAMA 2007; 28 (3), 299-308.)

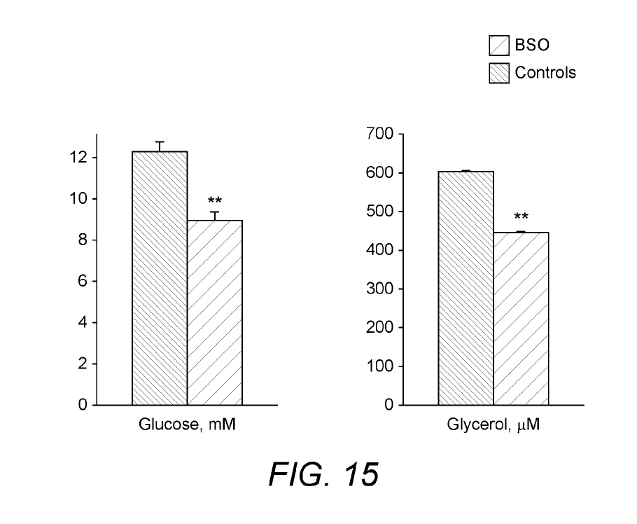

FIG. 15 compares plasma concentrations of glucose and glycerol in the BSO-treated and control mice. Glucose and glycerol are decreased in the BSO-treated mice. Lower glucose concentrations indicate better insulin sensitivity in the BSO-treated mice, meaning that the BSO-treated mice are likely to be more resistant to type II diabetes. Lower glycerol concentrations are consistent with the lower non-essential fatty acids, which in turn play a role in causing insulin resistance.

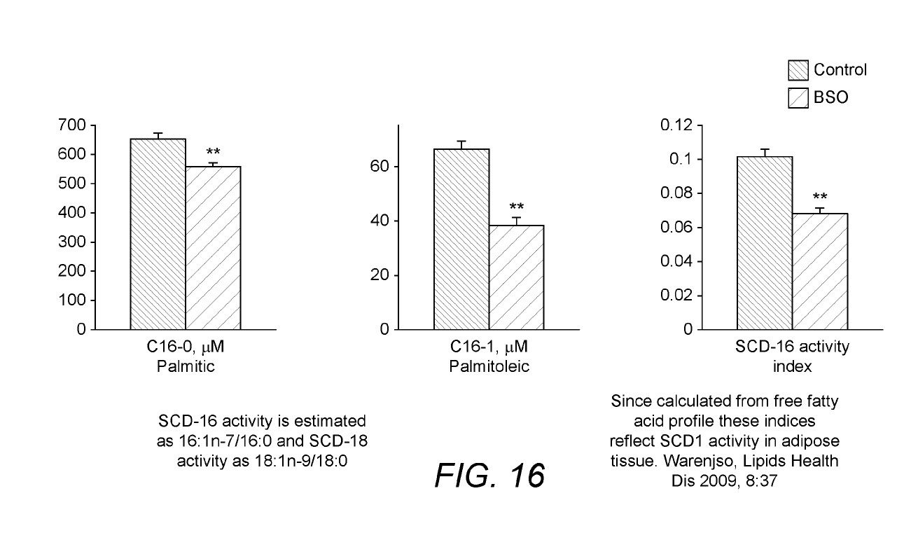

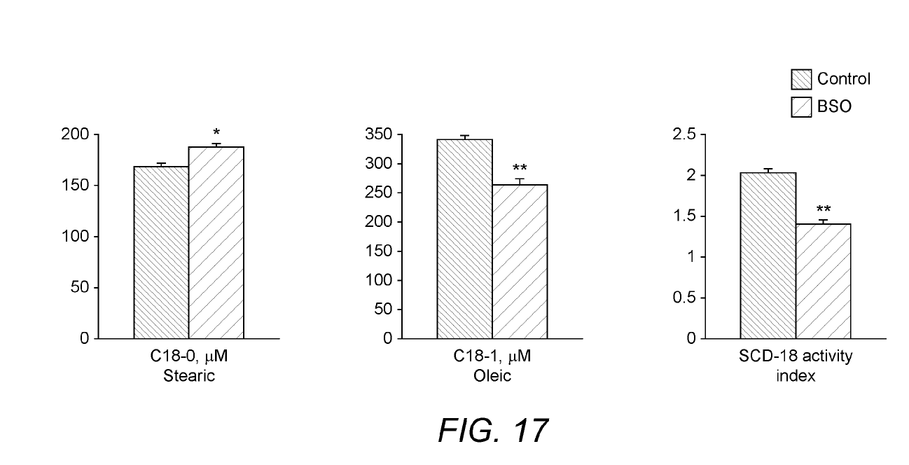

C16 fatty acid profiles in the plasma are compared in FIG. 16. Palmitic acid and palmitoleic acid concentrations are lower in the plasma of BSO-treated mice, the ratio of palmitoleic acid to palmitic acid also being lower in the BSO-treated mice, consistent with SCD inhibition. SCD inhibition is also demonstrated by the results in shown FIG. 17, for the C18 stearic and oleic acids, in that the oleic acid to stearic acid ratio is lower in the BSO-treated mice.

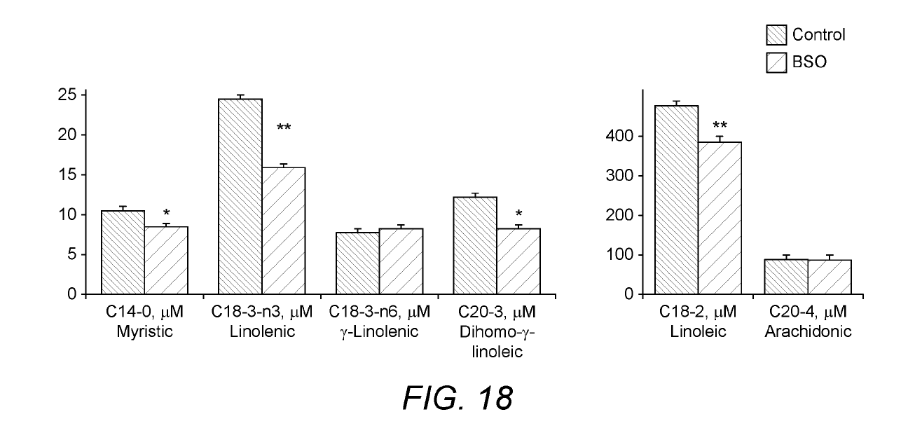

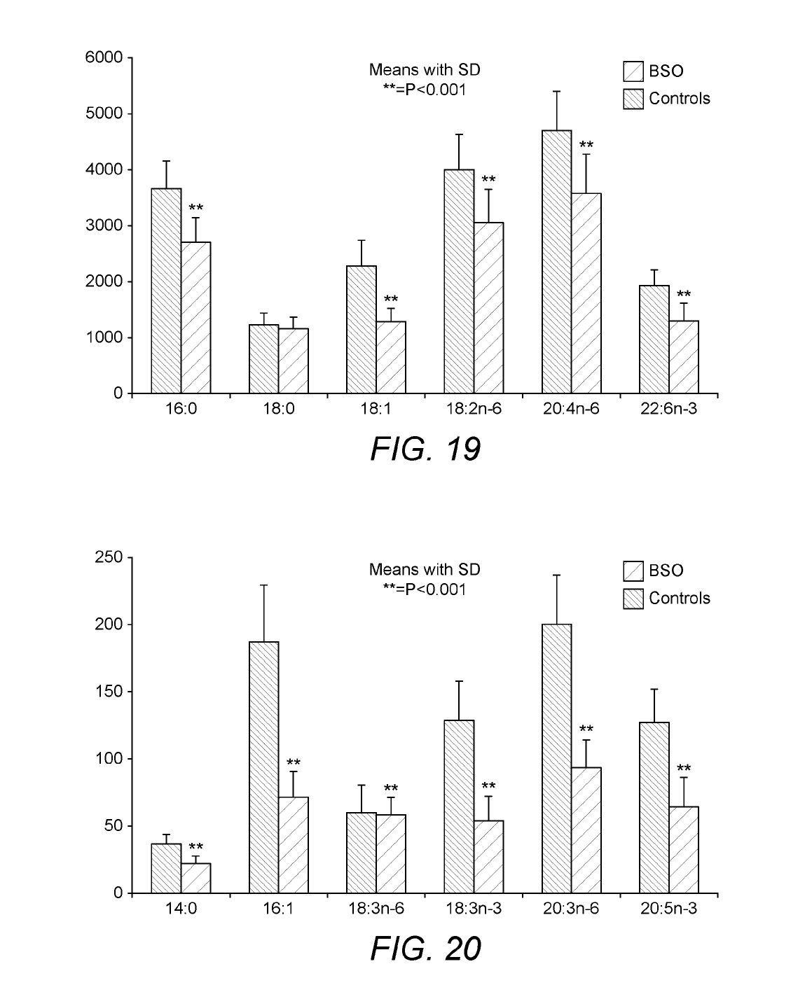

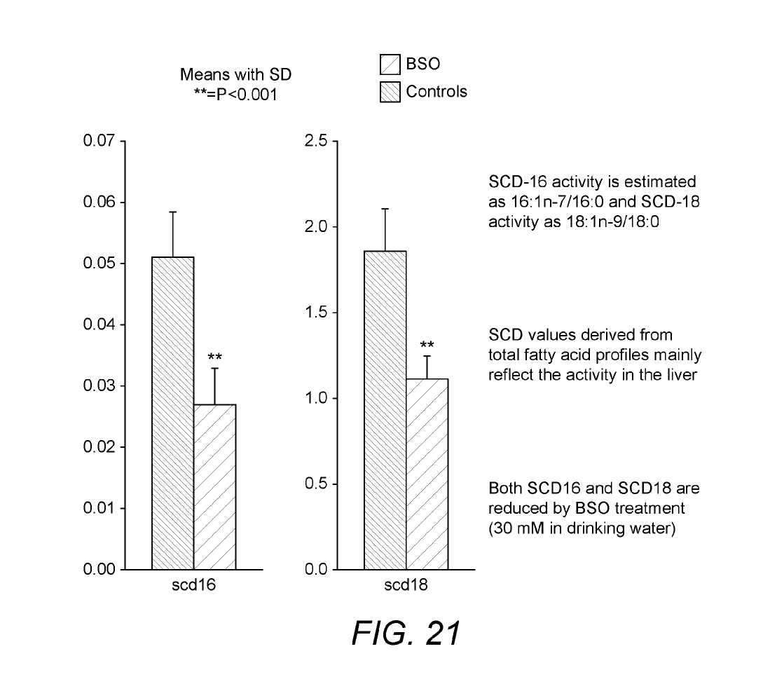

FIGS. 18 to 20 show further evidence of lower fatty acid plasma concentrations in BSO-treated mice for a number of C14 to C22 fatty acids, and FIG. 21 shows that the lower unsaturated/saturated fatty acid ratios are exhibited for total fatty acid concentrations as well as free fatty acid concentrations.

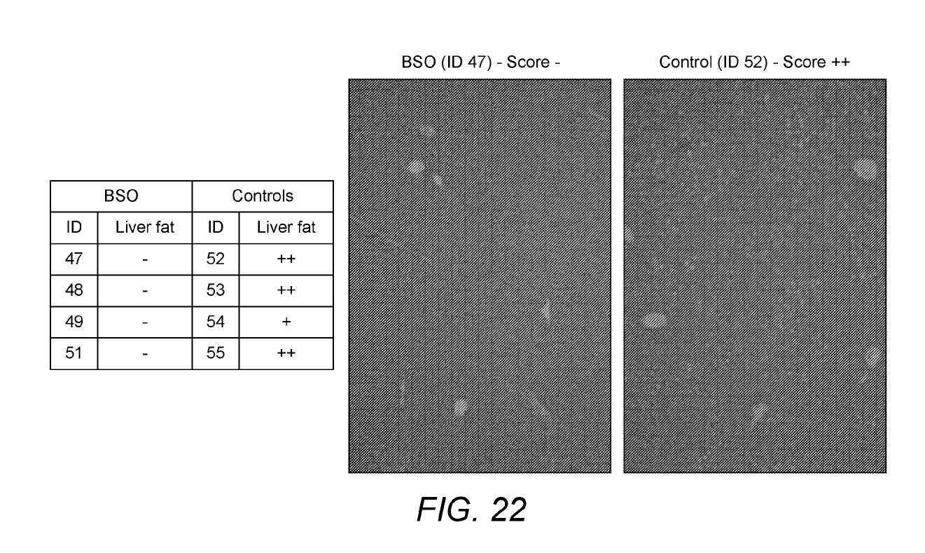

The effects of BSO on liver fat vacuolation are shown in FIG. 22, which compares liver cells of BSO-treated and control mice. Fat vacuolation is absent in the BSO-treated mice denoting protection against the non-alcoholic fatty liver disease induced by a high-fat diet, and which is a common complication of human obesity that predisposes to liver cirrhosis.

FIG. 23 shows further results of the effects of BSO on glucose and insulin levels. Thus, BSO-treated mice had lower glucose plasma levels, and lower insulin concentrations compared to the control. In addition, the HOMA insulin-resistance (IR) index is significantly lower in BSO-fed mice compared to control. The HOMA IR index is a calculation of insulin resistance based on fasting glucose and insulin plasma levels, the calculation being known to those skilled in the art. Leptin plasma levels are also shown. Higher plasma leptin levels correlate with increased obesity, hence these results further demonstrate the beneficial effects of BSO in reducing obesity.

FIG. 24 shows the effects of BSO on plasma and liver glutathione levels. In the plasma, both total glutathione (tGSH) and reduced glutathione (rGSH) are lower in the BSO-fed mice. The rGSH/tGSH ratio is similar. This is likely a result of inhibition of the enzyme GCL (glutamate cysteine ligase).

In the liver, the situation differs. Although tGSH levels are lower in the BSO-fed mice, the rGSH levels are similar, giving a higher rGSH/tGSH ratio in the BSO-fed mice. This suggests that the effects of BSO in the liver are more specific to the oxidized form of GSH, and indicates that the reduced form of GSH (the active antioxidant form) is not compromised by BSO at the doses required to facilitate weight loss. Such BSO doses are substantially lower than those used as an adjuvant to chemotherapy, hence it can be expected that BSO (and other compounds of Formula I) have low toxicity since levels of rGSH is preserved at such doses. This can also explain why BSO and other compounds of Formula I protect against liver pathology and fatty liver disease (e.g. non-alcoholic fatty liver disease and liver cirrhosis).

For the results presented in FIGS. 23 and 24, the experiments were carried out using the same conditions as described above (i.e. same mouse strain, mouse age, diet, and experimental design) except that the mice were treated for eight weeks with BSO, and the plasma for insulin, glucose, and leptin analysis was drawn after a 6-hour fast, after six weeks of BSO treatment.

1. A method of decreasing body fat or preventing or decreasing the accumulation of body fat in a subject in need thereof, comprising administering to the subject a therapeutically effective amount of a compound of formula I or pharmaceutically acceptable salts thereof, and wherein the compound inhibits the enzyme SCD; 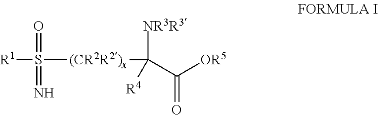
wherein;
R¹ is a C₁ to C₈ linear, branched or cyclic alkyl or alkenyl group having up to two double bonds, and optionally substituted with one or more groups selected from (i) one or more halogen atoms; (ii) alkoxy group of formula OR⁶; (iii) hydroxy; and (iv) carboxyl group of formula COOR⁷;
R, R^{2′}, R³ and R^{3′} are each independently and for each occurrence selected from (a) hydrogen; (b) C₁ to C₄ alkyl optionally substituted with one or more groups selected from (i) to (iii) above; (c) halogen; and (d) C₁-C₂ alkoxy; and where R² and R^{2′} are both selected from either (b) or (d), R² and R^{2′} can optionally be linked, and where R³ and R^{3′} are both selected from either (b) or (d), R³ and R^{3′} can optionally be linked;
R⁴ and R⁵ are each independently selected from hydrogen or C₁ to C₄ alkyl optionally substituted with one or more groups selected from (i) to (iii) above;
R⁶ is selected from linear, branched or cyclic alkyl optionally substituted with one or more halogen atoms;
R⁷ is selected from hydrogen and linear, branched or cyclic C₁ to C₄ alkyl; and
x is an integer from 1 to 3.

2. (canceled)

3. The method according to claim 1, wherein the subject is a mammal.

4. The method according to claim 1, wherein the subject is overweight or obese.

5. The method according to claim 4, wherein the subject is a human having a body mass index (BMI) of 25 kg/m² or more.

6. The method according to claim 1, wherein said method allows to avoid or reduce accumulation of body fat in the a subject.

7. The method according to claim 6, wherein said method allows to preferentially avoid or reduce white adipose tissue.

8. The method according to claim 1 wherein the subject is overweight or obese, or has one or more conditions associated with being obese or overweight selected from one or more of coronary heart disease, angina, high blood pressure, type 2 diabetes, glucose intolerance, insulin resistance, stroke, cancer, infertility, depression, liver disease, kidney disease, dementia, osteoarthritis, gastro-oesophageal reflux disease, and sleep apnoea.

9. The method according to claim 8, wherein the conditions associated with being obese or overweight are selected from insulin resistance and type 2 diabetes.

10. The method according to claim 8, wherein the subject has a liver disease.

11. The method according to claim 1, wherein in Formula I one or more of the following apply:
(a) R¹ is an optionally substituted C₃ to C₅ linear, branched or cyclic alkyl;
(b) R² and R²′ are independently and for each occurrence selected from hydrogen and optionally substituted C₁₋₂ alkyl; (c) R³ and R³′ are each independently selected from hydrogen and optionally substituted C₁₋₂ alkyl; and (d) R⁴ or R⁵ are each independently selected from hydrogen and optionally substituted C₁₋₂ alkyl.

12. The method according to claim 11, wherein in Formula I one or more of the following apply:
(a) R¹ is a C₄ alkyl;
(b) x is 2;
(c) All R² groups are hydrogen, and no more than two R²′ groups are other than hydrogen, selected from optionally substituted C₁₋₂ alkyl;
(d) R³ is hydrogen and R³′ is hydrogen or non-substituted C₁₋₂ alkyl;
(e) R⁴ and R⁵ are both hydrogen.

13. The method according to claim 12, wherein the compound of Formula I is buthione sulfoximine or a pharmaceutically acceptable salt thereof.

14. (canceled)

15. (canceled)
